(19) United States
(12) Patent Application Publication
(10) Pub. No.: US 2024/0198107 A1
(43) Pub. Date: Jun. 20, 2024

(54) SPLANCHNIC NEUROMODULATION FOR HEART CONDITIONS

(71) Applicant: Medtronic, Inc., Minneapolis, MN (US)

(72) Inventors: David John Miller, Austin, TX (US); Maneesh Shrivastav, Blaine, MN (US); ShaileshKumar V. Musley, Brooklyn Park, MN (US)

(21) Appl. No.: 18/389,093

(22) Filed: Nov. 13, 2023

Related U.S. Application Data
(60) Provisional application No. 63/433,704, filed on Dec. 19, 2022.

(51) Int. Cl. A61N 1/36; A61N 1/372

(52) U.S. Cl. CPC A61N 1/36139; A61N 1/36146; A61N 1/37247

(57) ABSTRACT

A therapeutic system and method are described. In one example, the system may include an implantable pulse generator configured to generate a current, a plurality of electrodes configured to apply the current to one or more splanchnic nerves of a patient, where each of the plurality of electrodes comprises at least one of an anode or a cathode, a processor, and memory. In some examples, the processor may receive sensor information describing subcutaneous nerve activity of the patient and adjust one or more parameters of the implantable pulse generator based on the subcutaneous nerve activity.

### CROSS-REFERENCE TO RELATED APPLICATIONS

This application claims the benefit of U.S. Provisional Application No. 63/433,704, filed on Dec. 19, 2022, which application is incorporated herein by reference in its entirety.

### BACKGROUND

The present disclosure is generally directed to therapeutic neuromodulation and relates more particularly to a stimulation/block therapy using an electrode device to affect cardiovascular conditions of a patient. The sympathetic nervous system provides major control of the vascular tone via the splanchnic nerves, and thus regulates fluid shifts into and out of the splanchnic compartment.

The splanchnic vascular compartment is the primary source of vascular capacity. Under normal conditions, sympathetic stimulation shifts blood from the splanchnic to the thoracic compartment to elevate cardiac preload and increase cardiac output. However, in patients with congestive heart failure, additional recruitment of preload has a detrimental effect and can make congestive heart failure symptoms worse and reduce exercise capacity. In heart failure, the ability of the splanchnic vascular compartment to store or buffer blood volume (capacitance) is impaired. The main regulatory system for the splanchnic vascular capacitance includes sympathetic fibers in the splanchnic nerves that control arterial and venous vascular tone. This makes the splanchnic vascular compartment and splanchnic nerves potential therapeutic targets in heart failure.

Functional redistribution of blood from unstressed to stressed vascular system compartments contributes to the rapid changes in systemic pulmonary venous pressures observed during acute heart failure. The shift in blood volume between the peripheral vascular compartments (i.e., the splanchnic vascular bed) and the central vascular compartment are major contributors to elevated left side filling pressures observed in heart failure patients. These indicators are also related to exertional capacity (e.g., during exercise), and therefore also proportionally impact quality of life and cardiovascular morbidity.

### BRIEF SUMMARY

### Example aspects of the present disclosure include:

A therapeutic system, comprising: an implantable pulse generator configured to generate a current; a plurality of electrodes configured to apply the current to one or more splanchnic nerves of a patient, wherein each of the plurality of electrodes comprises at least one of an anode or a cathode; a processor; and a memory storing data for processing by the processor, the data, when processed, causes the processor to: receive sensor information describing subcutaneous nerve activity of the patient; and adjust one or more parameters of the implantable pulse generator based on the subcutaneous nerve activity.

In one aspect, the sensor information is received from at least one sensor provided on or implanted in the patient.

In one aspect, the at least one sensor includes at least one of a wearable sensor, an implantable sensor, and an environmental sensor, and wherein the sensor information is received via a wireless communication signal from the at least one sensor.

In one aspect, the one or more parameters comprise at least one of frequency, peak current, duty cycle, and application duration.

In one aspect, the memory further stores data that, when processed by the processor, causes the processor to: estimate a sympathetic tone based on the sensor information describing the subcutaneous nerve activity.

In one aspect, the system further includes a user interface that allows at least one of the patient and a clinician to provide inputs to describe an aspect of the subcutaneous nerve activity.

In one aspect, the one or more splanchnic nerves includes at least one of a greater splanchnic nerve, a lesser splanchnic nerve, and a least splanchnic nerve.

In one aspect, the one or more parameters of the implantable pulse generator are adjusted to increase a nerve blocking applied at the one or more splanchnic nerves.

In one aspect, the one or more parameters of the implantable pulse generator are adjusted to decrease a nerve blocking applied at the one or more splanchnic nerves.

In one aspect, the one or more parameters of the implantable pulse generator are adjusted to increase a nerve stimulation applied at the one or more splanchnic nerves.

In one aspect, the one or more parameters of the implantable pulse generator are adjusted to decrease a nerve stimulation applied at the one or more splanchnic nerves.

In one aspect, the sensor information is received from at least one of the plurality of electrodes.

In another embodiment, a control subsystem is provided for use in a therapeutic system. In some examples, the control subsystem includes: a processor; and a memory storing data for processing by the processor, the data, when processed, causes the processor to: receive sensor information describing subcutaneous nerve activity of a patient; and determine an electrical signal applied by an implantable pulse generator to a splanchnic nerve of the patient is to be adjusted based on the subcutaneous nerve activity; and transmit a control signal to the implantable pulse generator thereby causing the implantable pulse generator to adjust the electrical signal applied to the splanchnic nerve.

In one aspect, the electrical signal comprises at least one of a blocking signal that provides a nerve block and a stimulation signal that provides a nerve stimulation to the splanchnic nerve.

In one aspect, the splanchnic nerve includes at least one of a greater splanchnic nerve, a lesser splanchnic nerve, and a least splanchnic nerve.

In one aspect, the sensor information is received from at least one sensor provided on or implanted in the patient.

In one aspect, the one or more parameters of the electrical signal are adjusted and the one or more parameters include at least one of frequency, peak current, duty cycle, and application duration.

In another embodiment, a method is provided that includes: determining a first state of a therapy for a patient, wherein the first state of the therapy includes providing electrical signals to one or more splanchnic nerves of the patient; receiving sensor information from one or more sensors; based on the received sensor information, determining a subcutaneous nerve activity associated with the patient; and based on the subcutaneous nerve activity, adjusting one or more parameters of the electrical signals to change the therapy from the first state to a second state.

In one aspect, the first state of the therapy further includes a unilateral ablation of at least a portion of the splanchnic nerve.

In one aspect, the electrical signals are applied bilaterally to the one or more splanchnic nerves and wherein adjusting the one or more parameters comprises changing at least one of a frequency, peak current, duty cycle, and application duration for the electrical signals.

In another embodiment, a system is provided that includes: a sensor that monitors one or more conditions of a patient or an environment surrounding a patient and that generates sensor information based thereon; an implantable pulse generator configured to generate a current; a plurality of electrodes configured to apply the current to one or more splanchnic nerves of the patient; a processor; and a memory storing data for processing by the processor, the data, when processed, causes the processor to: receive the sensor information from the sensor, wherein at least some of the sensor information describes subcutaneous nerve activity of the patient; and adjust one or more parameters of the implantable pulse generator based on the subcutaneous nerve activity.

In one aspect, the sensor includes an implantable sensor.

In one aspect, the sensor includes a wearable sensor.

In one aspect, the sensor includes an environmental sensor.

In one aspect, the one or more parameters of the implantable pulse generator are adjusted and change at least one of an electrical block and electrical stimulation applied to the one or more splanchnic nerves based on the sensor information.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a diagram of a system according to at least one embodiment of the present disclosure;

FIG. 2 is a diagram of an ablation tool used according to at least one embodiment of the present disclosure;

FIG. 3 is block diagram of system components used to provide therapeutic treatment according to at least one embodiment of the present disclosure;

FIG. 4 is a flowchart illustrating a therapeutic method according to at least one embodiment of the present disclosure; and

FIG. 5 is a flowchart illustrating another therapeutic method according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, different lead configurations are described and may be designed so as to optimize current density and provide the current (e.g., stimulation and/or blocking) at different angles to a target anatomical element. The lead may comprise electrode(s) that may be selected to comprise an anode or a cathode. In some embodiments, the electrode(s) may comprise a combination of anodes or cathodes and in other embodiments the electrode(s) may comprise all anodes or cathodes. The electrode(s) may also be selected to be active or inactive.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., random-access memory (RAM), read-only memory (ROM), electrically erasable programmable ROM (EEPROM), flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia Geforce RTX 2000-series processors, Nvidia Geforce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

Recent research has shown that thermal destruction of the greater splanchnic nerve restores the sympathetic-parasympathetic balance and, in doing so, redistributes a portion of the blood volume from the thoracic compartment to the splanchnic compartment. Bi-lateral splanchnic ablation is not tolerated in humans due to excessive side effects, so unilateral splanchnic ablation is currently under investigation. Splanchnic nerve ablation can be performed using a percutaneous approach with a needle. Percutaneous splanchnic nerve ablation can be performed as a method to relieve intractable pain from pancreatic cancer or other causes. Percutaneous splanchnic nerve ablation utilizes a long needle navigated around the bones and other structures of the spine. The splanchnic nerve can also be ablated using a catheter within nearby Azygous veins.

Aspects of the present disclosure provide therapeutic mechanisms designed to treat heart conditions. While embodiments of the present disclosure will be described in connection with treatment of heart conditions and other cardiac improvements, it should be appreciated that embodiments of the present disclosure are not limited to treating heart conditions. Rather, therapeutic approaches depicted and described herein can be used to treat other conditions, which may or may not relate to cardiac conditions. In some embodiments, the therapeutic mechanisms proposed herein aim to modulate instead of ablating the splanchnic nerve. Other aspects of the present disclosure provide therapeutic mechanisms of modulation that supplement nerve ablation.

Current solutions permanently ablate the nerve on one side. While this approach has a clinical benefit, a better outcome can be obtained with the embodiments provided in the present disclosure. Specifically, more potent therapeutic mechanisms are proposed herein that enable treatment of the splanchnic nerve on both sides instead of one side thereof. Additionally, more intelligent therapeutic mechanisms are proposed herein that utilize a closed-loop system that intermittently blocks and/or stimulates one or more nerves based on feedback received from inputs or sensors.

Peripheral nerves can be partially or completely blocked using high-frequency alternating current from nearby electrodes. This high-frequency alternative current can be delivered by an implanted lead having two or more electrodes. According to at least some embodiments of the present disclosure, neuromodulation could be unilateral or bilateral, or could be combined with ablation by ablating one side and blocking the other side.

In some embodiments, the ablation may be delivered with a catheter using radiofrequency energy, microwave energy, and/or pulsed-field energy. In some embodiments, the ablation may also use cryoablation to freeze the nerve(s). Alternatively or additionally, chemical ablation may be employed.

To temporarily block the splanchnic nerve, a frequency of between approximately 5-KHz and 50 KHz could be delivered to or near a splanchnic nerve (e.g., the greater splanchnic nerve, the lesser splanchnic nerve, and/or the least splanchnic nerve). The leads used to deliver this energy could be routed to the site through the soft tissue and/or through the vertebral body.

In addition to blocking the splanchnic nerve with kHz frequency signal(s), the same leads could stimulate the splanchnic nerve(s) to increase the amount of blood in the thoracic compartment and decrease the volume in the splanchnic compartment. This could be desired in some situations like low blood pressure, orthostatic low-blood pressure, or hypovolemia.

In some embodiments, an implantable pulse generator is provided and is configured to deliver the high-frequency stimulation to block the splanchnic nerve. The implantable pulse generator, in some embodiments, may have a primary cell and/or a rechargeable cell as a power source.

There are situations where more or less blood is desired in the splanchnic organs. This can be based on retained fluids, patient activity, time of day, consumption of food, and other activities. In accordance with at least some embodiments, these activities could be automatically sensed by a device (e.g., one or more sensors) implanted in the patient or external to the patient and communicated to the internal stimulation device, which controls operation of the pulse generator. The signals could also come from existing implantable monitoring devices, such as an implantable cardiac monitor. The signals could also come from a wearable and removeable device, such as a smart watch, a smart ring, a heart rate monitor, etc.

Other examples of sensor devices that could be used to sense patient activities include, without limitation, accelerometers, glucose sensors, heart rate sensors, muscular electrical signal sensors, combinations thereof, and the like. There could also be inputs for the patient and/or clinician to modify one or more settings of the implantable pulse generator.

One aspect of the present disclosure is to provide the ability to improve on unilateral splanchnic ablation by affecting the splanchnic nerve on both sides, when needed. Such an approach allows for more potent and adaptive control of the blood in the splanchnic reservoir.

Another aspect of the present disclosure is to provide the ability to automatically or on-demand change splanchnic neuromodulation. Such changes can impact the distribution of blood between splanchnic and thoracic compartments.

In some embodiments, a clinical solution like the one described herein has the potential to provide a meaningful improvement to millions of patients and reduce general healthcare costs associated with treating or preventing cardiac conditions.

In some embodiments, the implantable pulse generator may be provided with at least two bipolar leads. Therapy provided by the implantable pulse generator (blocking and/or stimulation) may be modulated by feedback mechanisms, which may include automated feedback mechanisms and/or manual feedback provided via a patient and/or clinician.

In some embodiments, feedback may be based on symptoms of decompensation. Illustrative, but non-limiting examples of symptoms of decompensation may include dyspnea, fluid retention, and/or fatigue. Some of these signs may result in progressive weight gain. Some or all of these symptoms may be used to modulate therapy provided by the implantable pulse generator. Dyspnea (labored breathing) may be measured via manual user inputs, meaning that the dyspnea is measured by the patient and/or clinician and input to the feedback loop. Fluid retention may correspond to a weakened condition of the heart, which causes less blood pumped through the kidneys, resulting in water retention that manifests in swollen legs, ankles, and abdomen. This can also cause increased need to urinate and can be measured by impedance (e.g., at a tip of the implanted electrode). Much like dyspnea, fatigue can also be measured based on user input (patient and/or clinician). Activity can be measured with one or more sensors implanted in the patient. Such implanted sensors may be integral with the pulse generator or may be provided in a separate implanted device. Patient weight may also be measured. If patient weight (HFrEF) dramatically increases (e.g., 4-5 lbs per week) due to increased water conditions, then the feedback loop may adjust the pulse generator to change the blocking and/or stimulation applied. It should be appreciated that one, some, or all of these symptoms can be correlated to the degree of therapy modulation.

As a non-limiting example, if one or more symptoms (e.g., reduced activity compared to baseline, increase in fluid retention, reported tiredness, reported labored breathing) are detected in the feedback loop, then the therapy intensity may be increased. Increases to therapy applied by the implantable pulse generator may include increasing a duty cycle of stimulation and/or increasing the electrical block of the greater splanchnic nerve.

As another non-limiting example, if one or more symptoms (e.g., increased activity as compared to baseline, no change in fluid retention, no reported tiredness, orthostatic hypotention, reported normal breathing) are detected in the feedback loop, then the therapy intensity may be reduced. Reductions to therapy applied by the implantable pulse generator may include reducing the duty cycle of stimulation and/or decreasing the electrical block of the greater splanchnic nerve.

In some embodiments, feedback may be obtained based on subcutaneous nerve activity. The sympathetic tone is well recognized in the control of cardiovascular systems. Sympathetic activity is increased in heart failure and is responsible for initiation and development of this disease. The subcutaneous space is richly innervated with sympathetic neurons. In accordance with at least some embodiments, subcutaneous nerve activity may be recorded to estimate sympathetic tone. Alternatively or additionally, nerve activity can be recorded via cuff electrodes on the nerves itself or subcutaneously via one or more sensors provided on/in the patient. Thus, recording subcutaneous nerve activities by an implanted device might offer an indirect, yet less invasive way, to monitor cardiac sympathetic activity in vivo.

Subcutaneous nerve activity can, therefore, be used to predict decompensation in HFpEF patients. The ability of the splanchnic vascular compartment to store or buffer blood volume is impaired in these patients. The main regulatory system for the splanchnic vascular capacitance includes sympathetic fibers in the splanchnic nerves that control arterial and venous vascular tone. This makes the splanchnic vascular compartment and greater splanchnic nerves potential therapeutic targets in HFpEF patients. Based on subcutaneous nerve activity, modulation of splanchnic nerve by electrical blockage is considered.

In some embodiments, feedback may be obtained from external and/or implantable data sources (e.g., sensor(s)). Examples of sensors and/or data sources that can be used in the feedback loop to modulate the therapy include, without limitation:
wearable devices such as a mobile cardiac outpatient telemetry (MCOT), continuous glucose monitor, smartwatch, smart ring, activity monitor, or impedance monitor
an implantable subcutaneous measurement device
an implantable cardioverter defibrillator, a pacemaker, or neurostimulator
self-reported patient symptoms
clinician input or feedback
application(s) that record health information
global positioning systems, beacon systems, or the like that can detect proximity to a restroom (e.g., as a proxy for increased urination due to frequency of visits)

In some embodiments, one or more therapies described herein may be applicable to patients with heart failure exhibiting preserved or reduced ejection fraction. As described herein, one or more neuromodulation techniques are provided for treatment of cardiac conditions. For example, neuromodulation techniques may generally include using a device (e.g., including at least an implantable pulse generator) to provide electrical stimulation (e.g., electrical pulses/impulses) on one or more trunks of the splanchnic nerve. In some examples, the device may provide stimulation and/or blocking (e.g., using the device). For example, the greater splanchnic nerve may be electrically blocked (e.g., down-regulated) by delivering a high frequency stimulation (e.g., of a range about 1 kilohertz (kHz) to 50 kHz, or more specifically about 5 kilohertz (kHz)). Additionally or alternatively, the greater or lesser splanchnic nerve may be electrically stimulated (e.g., up-regulated) by delivering a low frequency stimulation (e.g., a square wave at a range of about 0.1 Hz to about 20 Hz, or more specifically, about 1 Hz).

As previously described, the electrical blocking and/or stimulation may be provided using electrodes. However, conventional electrode designs may not conform to or otherwise fit to an anatomical element, and may need multiple implantation procedures for insertion. Further, typical electrode designs are restrained to an initial design of the electrode(s) and may not enable customization of the electrode(s).

Thus, embodiments of the present disclosure provide technical solutions to (1) improve patient outcomes for those patients exhibiting heart conditions, (2) update therapies in real-time based on one or more feedback loops, and (3) provide optimized and, possibly, patient-specific therapies using electrical blocking, electrical stimulation, and/or ablation techniques.

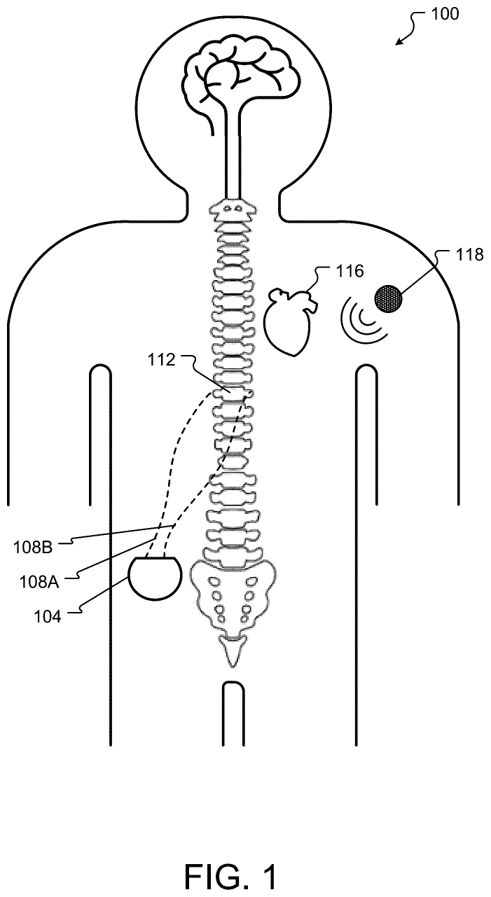

Turning to FIG. 1, a diagram of a system 100 according to at least one embodiment of the present disclosure is shown. The system 100 may be used to provide one or more therapies for a patient and/or carry out one or more other aspects of one or more of the methods disclosed herein. For example, the system 100 may include at least a device that is capable of providing a stimulation/blocking therapy that corrects or adjusts one or more functions of the splanchnic nerve for purposes of supporting improved cardiac performance. In some examples, the device may be referred to as an implantable pulse generator 104. More specifically, the implantable pulse generator 104 may be configured to generate a current or electrical signal. Additionally, the system 100 may include one or more wires 108A, 108B (e.g., leads) that provide a connection between the device implantable pulse generator 104 and nerves of the patient for enabling the stimulation/blocking therapy.

As described previously, neuromodulation techniques (e.g., technologies that act directly upon nerves of a patient, such as the alteration, or “modulation,” of nerve activity by delivering electrical impulses or pharmaceutical agents directly to a target area) may be used for assisting in treatments for different diseases, disorders, or ailments of a patient, such as cardiac ailments. In some embodiments, the implantable pulse generator 104 may provide electrical stimulation to one or more anatomical elements 112 for purposes of impacting one or more other anatomical elements 116. In the illustrated and described examples, each of the wires 108A, 108B may have one, two, three, four, or more electrodes thereon configured to deliver electrical signals, stimulation, or pulses to the one or more anatomical elements 112. Delivery of the electrical signals, stimulation, or pulses to the anatomical element(s) 112 may provide one or more therapeutic conditions for the other anatomical elements. In some examples, the anatomical element 112 to which the electrical signals, stimulation, or pulses are delivered may correspond to one or more splanchnic nerves (e.g., the greater splanchnic nerve, the lesser splanchnic nerve, and/or the least splanchnic nerve). Delivery of the electrical signals, stimulation, or pulses to the splanchnic nerves may provide a therapy to the other anatomical element 116, which may include the heart 116 or other parts of the cardiovascular system.

In some examples, the one or more wires may include at least a first wire 108A and a second wire 108B connected to or implanted near splanchnic nerves. Splanchnic organs (e.g., organs which may be connected to or have at least some functionality associated with the splanchnic nerves) include: stomach, spleen, pancreas, and intestines. Splanchnic organs contain approximately between 20% and 30% of the total blood volume. Veins that move blood between splanchnic organs may be highly compliant (>20:1) and have a smooth muscle layer with dense autonomic innervation. The splanchnic compartment acts as primary blood reservoir from which blood can be recruited in and out based on need. Patients with heart failure or some form of cardiovascular disease may have sympathetic hyperactivation resulting in impaired response. Implanting one or more electrodes from the one or more wires 108A, 108B near a splanchnic nerve can help support movement of blood between splanchnic organs.

Examples of implantable wires having one or more electrodes are depicted and described in U.S. Patent Publication No. 2010/0114254 and U.S. Pat. No. 8,694,123, each of which are incorporated herein by reference in their entirety. It should be appreciated that any suitable electrode configuration can be used for the wires 108A, 108B to delivery electrical signals, stimulation, or pulses to the splanchnic nerve(s).

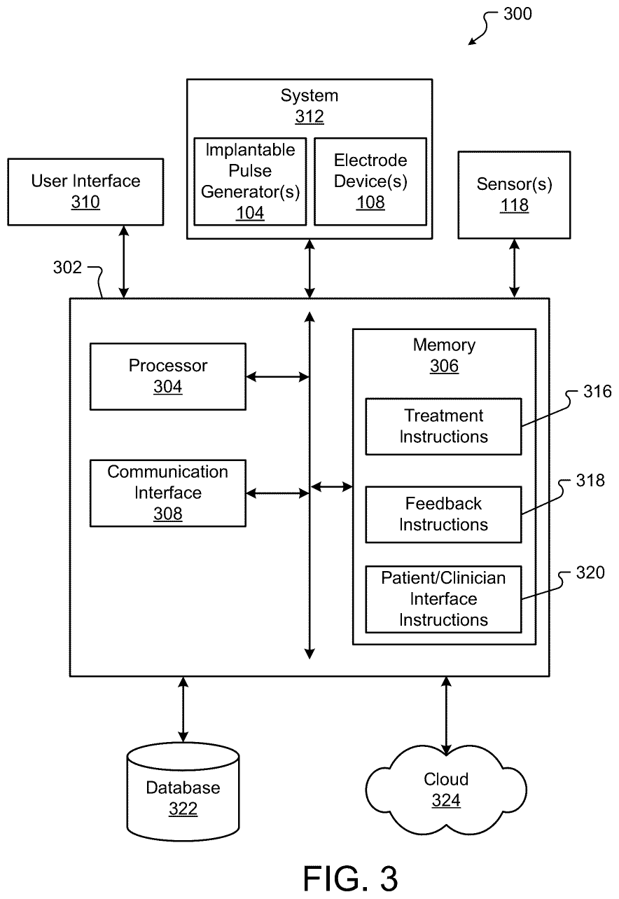

Additionally, while not shown, the system 100 may include one or more processors (e.g., one or more DSPs, general purpose microprocessors, graphics processing units, ASICs, FPGAs, or other equivalent integrated or discrete logic circuitry) shown and described in FIG. 3 that are programmed to carry out one or more aspects of the present disclosure. Alternatively or additionally, the one or more processors in the system may be configured to execute instructions stored in one or more computer memory devices, which may be separate from the processors or integrated with the processors. In some examples, the one or more processors may include a memory or may be otherwise configured to perform the aspects of the present disclosure. For example, the one or more processors may provide instructions to the implantable pulse generator 104, or other components of the system 100 not explicitly shown or described with reference to FIG. 1 for providing the stimulation/blocking therapy. In some examples, the one or more processors may be part of the implantable pulse generator 104 or part of a control unit for the system 100 (e.g., where the control unit is in communication with the implantable pulse generator 104 and/or other components of the system 100).

The system 100 or similar systems may be used, for example, to carry out one or more aspects of any of the methods 400, 500 described herein. The system 100 or similar systems may also be used for other purposes. It will be appreciated that the human body has more than one splanchnic nerves (or nerve branches) and the stimulation and/or blocking therapies described herein may be applied to one or more splanchnic nerve branches, which may reside at any location of a patient (e.g., lumbar, thoracic, etc.). Further, a sequence of stimulations and/or blocking therapies may be applied to different nerves. For example, a low frequency stimulation may be applied to a first nerve and a high frequency blockade may be applied to a second nerve.

FIG. 1 also illustrates a sensor 118, which may include one or more sensors 118 that are provided on the patient. The sensor(s) 118 may include implantable sensor(s), wearable sensor(s), and/or environmental sensor(s). The sensor(s) 118 as will be described in further detail herein, may provide sensor information describing one or more aspects of the patient's current or historical health conditions. The sensor information may be provided as feedback to a therapeutic system that modulates, controls, or adjusts the electrical signals, stimulation, and/or pulses provided by the implantable pulse generator 104. In some embodiments, the sensor information may be provided directly to the implantable pulse generator 104, which is configured to adjust or modulate electrical signals applied to one or more splanchnic nerves based on the sensor information. In some embodiments, the sensor information may be provided to an external controller, which may or may not be provided on the patient, which determines a therapy to apply to the patient and then instructs the implantable pulse generator 104 to generate the appropriate electrical signals, stimulations, and/or pulses. As will be described herein, the combination of the implantable pulse generator 104 and sensor(s) 118 help to create a feedback control loop that can selectively modify and tune therapies applied to the splanchnic nerve for purposes of treating one or more cardiac conditions.

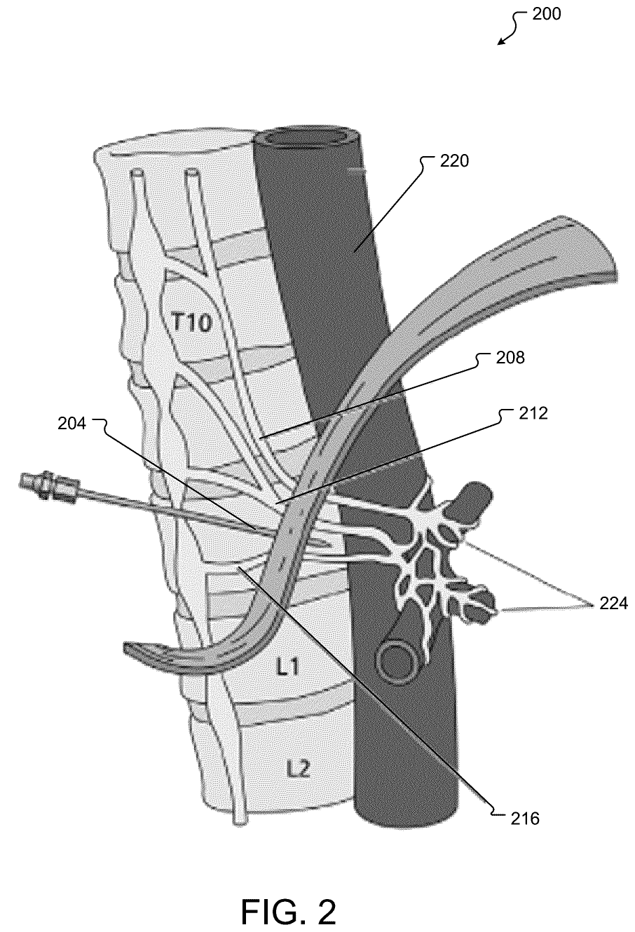

Turning now to FIG. 2, additional details of a system 200 used to ablate one or more splanchnic nerves or tissue near one or more splanchnic nerves will be described in accordance with at least some embodiments of the present disclosure. The system 200 is shown to include an ablation tool 204 that is configured to be positioned near or proximate one or more splanchnic nerves. The ablation tool 204 may be configured to apply heat, RF energy, chemicals, or the like for purposes of ablating tissue on, around, or near one or more splanchnic nerves. The anatomy around the splanchnic nerve area may include the greater splanchnic nerve 208, the lesser splanchnic nerve 212, the least splanchnic nerve 216, the aorta 220, and the celiac plexus 224. The splanchnic nerves are generally found the lower thoracic region (e.g., near T10) and/or the upper lumbar region (e.g., near L1).

In some embodiments, therapies provided herein may include a combination of ablating, blocking, and stimulating one or more splanchnic nerves. In some embodiments, unilateral greater splanchnic nerve 208 ablation may be combined with unilateral or contralateral active neuromodulation techniques (e.g., electrical stimulation and/or electrical blocking). Although not depicted, it should be appreciated that one or both of the wires 108A, 108B may be implanted on or near any of the greater splanchnic nerve 208, the lesser splanchnic nerve 212, and the least splanchnic nerve 216. Electrical signals (e.g., stimulation and/or blocking signals) may be delivered to one, some, or all of the splanchnic nerves 208, 212, 216.

Referring now to FIG. 3, additional details of a system 300 used to provide and modulate therapies for a patient will be described. The system 300 is shown to include a control subsystem 302 as well as a delivery subsystem 312. The delivery subsystem 312 may include one or more components of system 100 depicted and described in connection with FIG. 1. Illustratively, the delivery subsystem 312 may include the implantable pulse generator 104 and wires 108A, 108B, which may include one or more electrode devices.

The control subsystem 302 may be in communication with the delivery subsystem 312 as well as the sensor(s) 118. The control subsystem 302, in some embodiments, may be provided as part of the implantable pulse generator 104. In some embodiments, the control subsystem 302 may be provided separate from the implantable pulse generator 104. As will be described, the control subsystem 302 may include components that create a closed feedback loop that supports treatment of one or more heart conditions. The control subsystem 302 may utilize information from one or more sensor(s) 118 (e.g., sensor information) as well as information from external data sources (e.g., a database 322). The control subsystem 302 may also be configured to communicate with external devices using direct machine-to-machine communications and/or via a communication network, such as the cloud network 324.

The control subsystem 302 may be provided as one or more computing devices, which may correspond to server-type devices and/or client devices. Alternatively or additionally, the control subsystem 302 may be provided on a user device, such as a smartphone, wearable device, an implantable device, a personal computer, a tablet, or the like. Systems according to other embodiments of the present disclosure may comprise greater or fewer components than the system 300. For example, the system 300 may not include one or more components of the control subsystem 302, the delivery subsystem 312, the database 322, and/or the cloud 324.

The delivery subsystem 312 may communicate with the control subsystem 302 to receive instructions such as treatment instructions 316 for applying a current to the anatomical element 112. The delivery subsystem 312 may also provide data (such as data received from an electrode device capable of recording data), which may be used to optimize the electrodes of the electrode device and/or to optimize parameters of the current generated by the implantable pulse generator 104.

The control subsystem 302 illustratively comprises a processor 304, a memory 306, a communication interface 308, and a user interface 310. Control subsystems 302 according to other embodiments of the present disclosure may comprise greater or fewer components than the depicted and described herein.

The processor 304 of the control subsystem 302 may be any processor described herein or any similar processor. The processor 304 may be configured to execute instructions stored in the memory 306, which instructions may cause the processor 304 to carry out one or more computing steps utilizing or based on data received from the sensor(s) 118, the system 312, the database 322, and/or the cloud 324.

The memory 306 may be or comprise RAM, DRAM, SDRAM, other solid-state memory, any memory described herein, or any other tangible, non-transitory memory for storing computer-readable data and/or instructions. The memory 306 may store information or data useful for completing, for example, any step of any method described herein, or of any other methods. The memory 306 may store, for example, instructions and/or machine learning models that support one or more functions of the system 300. For instance, the memory 306 may store content (e.g., instructions and/or machine learning models) that, when executed by the processor 304, enable the processor 304 to perform one or more functions described herein. Illustratively, and without limitation, the memory 306 may store treatment instructions 316, feedback instructions 318, and/or user interface instructions 320.

The treatment instructions 316, when executed by the processor 304, may enable the control subsystem 312 to interact with the delivery subsystem 312. The treatment instructions 316 may be configured to provide control signals or inputs to the delivery subsystem 312. More specifically, the treatment instructions 316 may be configured to control and/or adjust operational parameters of the implantable pulse generator 104.

The treatment instructions 316 may also be configured to determine preferred and/or optimal treatments for a patient based on current patient conditions as measured by sensor(s) 118 and as processed by the feedback instructions 318. Said another way, the treatment instructions 316 may cooperate with the feedback instructions 318 to provide a feedback loop that adjusts or modulates treatment provided by the delivery subsystem 312. The feedback instructions 318, when executed by the processor 304, may enable the processor 304 to receive sensor information from the sensor(s) 118, analyze the sensor information, and determine if one or more adjustments to a currently-provided therapy should be made. If the feedback instructions 318 determine that adjustments to a current therapy should be made, then the feedback instructions 318 may provide information describing the change to the treatment instructions 316, which, in response thereto, instruct the implantable pulse generator 104 to adjust the treatment. In some embodiments, the feedback instructions 318 and treatment instructions 316 may cooperate for purposes of determining an initial therapy to apply to a patient. In other words the instructions 316, 318 may be configured to work together when determining initial therapy parameters and/or adjustments to therapy parameters.

The user interface instructions 320, when executed by the processor 304, may enable a patient and/or clinician to interact with the control subsystem 302. Specifically, and without limitation, the user interface instructions 320 may be configured to enable operation of the user interface 310, to receive user inputs from the user interface 310, and to provide inputs or commands to the treatment instructions 316 and/or feedback instructions 318 based on the user inputs received via the user interface 310. As an example, a patient may be allowed to provide inputs describing a current state of their health (e.g., fatigued, not fatigued, short of breath, not short of breath, etc.). the user input received from the patient may be provided to the feedback instructions 318 to determine if changes to a current therapy are desired or required. As another example, a clinician may be allowed to provide inputs describing bounds (e.g., upper or lower bounds) for one or more therapeutic parameters. Illustratively and without limitation, the clinician may provide inputs via the user interface 310 that define upper or lower bounds on current that is applied by the implantable pulse generator, a frequency of signals applied, a duty cycle used, conditions under which to change a therapy, etc.

Content stored in the memory 306, if provided as in instruction, may, in some embodiments, be organized into one or more applications, modules, packages, layers, or engines. Alternatively or additionally, the memory 306 may store other types of content or data (e.g., machine learning models, artificial neural networks, deep neural networks, etc.) that can be processed by the processor 304 to carry out the various method and features described herein. Thus, although various contents of memory 306 may be described as instructions, it should be appreciated that functionality described herein can be achieved through use of instructions, algorithms, and/or machine learning models. The data, algorithms, and/or instructions may cause the processor 304 to manipulate data stored in the memory 306 and/or received from or via the system 312, the database 322, and/or the cloud 324.

The control subsystem 302 may also comprise a communication interface 308. The communication interface 308 may be used for receiving data (for example, data from an electrode device of the delivery subsystem 312 capable of recording data) or other information from an external source (such as sensor(s) 118, the database 322, the cloud 324, and/or any other system or component not part of the system 300), and/or for transmitting instructions, images, or other information to an external system or device (e.g., another control subsystem 302, the delivery subsystem 312, the database 322, the cloud 324, and/or any other system or component not part of the system 300). The communication interface 308 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 308 may be useful for enabling the control subsystem 302 to communicate with one or more other processors (e.g., in the implantable pulse generator 104) or computing devices, whether to reduce the time needed to accomplish a computing-intensive task or for any other reason.

The control subsystem 302 may also comprise one or more user interfaces 310. The user interface 310 may be or comprise a keyboard, mouse, trackball, monitor, television, screen, touchscreen, and/or any other device for receiving information from a user and/or for providing information to a user. The user interface 310 may be used, for example, to receive a user selection or other user input regarding any step of any method described herein. Notwithstanding the foregoing, any required input for any step of any method described herein may be generated automatically by the system 300 (e.g., by the processor 304 or another component of the system 300) or received by the system 300 from a source external to the system 300. In some embodiments, the user interface 310 may be useful to allow a patient, surgeon, clinician, or the like to modify instructions to be executed by the processor 304 according to one or more embodiments of the present disclosure, and/or to modify or adjust a setting of other information displayed on the user interface 310 or corresponding thereto.

Although the user interface 310 is shown as being separated (e.g., outside of) the control subsystem 302, in some embodiments, the control subsystem 302 may utilize a user interface 310 that is included in the control subsystem 302. In some embodiments, the user interface 310 may be located proximate one or more other components of the control subsystem 302, while in other embodiments, the user interface 310 may be located remotely from one or more other components of the control subsystem 302. In some embodiments, the user interface 310 may be provided as part of a mobile communication device (e.g., smartphone) carried by the patient and/or a clinician. Communications between the user interface 310 and control subsystem 302 may be achieved using wireless and/or wired communication signals.

The database 322 may store information such as patient data, results of a stimulation and/or blocking procedure, stimulation and/or blocking parameters, current parameters, electrode parameters, etc. The database 322 may be configured to provide any such information to the control subsystem 302 or to any other device of the system 300 or external to the system 300, whether directly or via the cloud 324. In some embodiments, the database 322 may be or comprise part of a hospital image storage system, such as a picture archiving and communication system (PACS), a health information system (HIS), and/or another system for collecting, storing, managing, and/or transmitting electronic medical records.

The cloud 324 may be or represent the Internet or any other wide area network. The control subsystem 302 may be connected to the cloud 324 via the communication interface 308, using a wired connection, a wireless connection, or both. In some embodiments, the control subsystem 302 may communicate with the database 322 and/or an external device (e.g., a computing device) via the cloud 324.

The system 300 and other systems may be used, for example, to carry out one or more aspects of any of the methods, which will now be described in further detail. The system 300 or similar systems may also be used for other purposes.

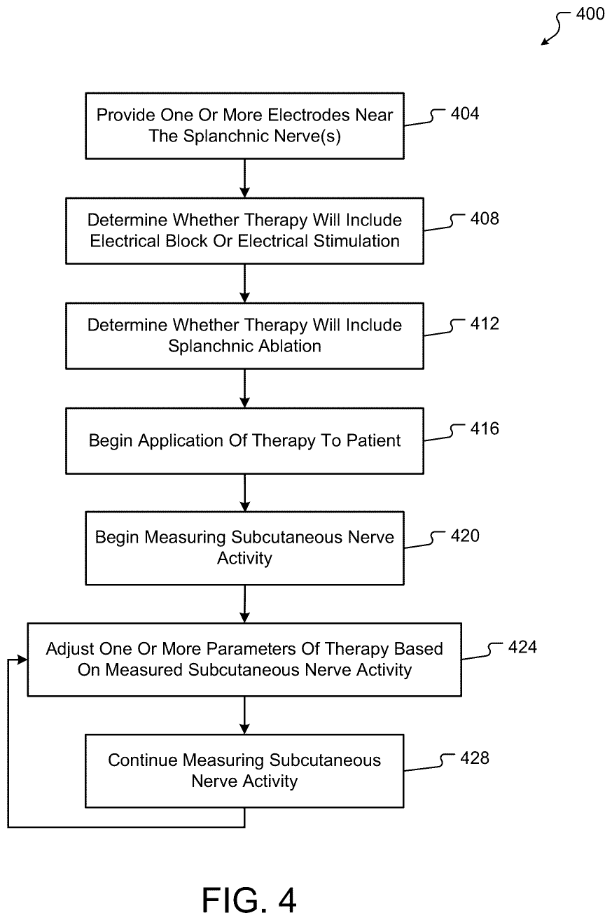

Referring now to FIG. 4, a first method 400 will be described in accordance with at least some embodiments of the present disclosure.

The method 400 begins by providing one or more electrodes near one or more splanchnic nerves of a patient (step 404). As described above, the electrodes may be provided on or near a splanchnic nerve or multiple splanchnic nerves. Electrodes may be placed in a suitable position via implantation, as an example. In some embodiments, two or more wires 108A, 108B are implanted, each having two or more electrodes used to delivery electrical therapies (e.g., electrical stimulation, electrical blocking, or the like) to the splanchnic nerve(s) and/or measure neural activity at or near the splanchnic nerve(s).

The method 400 may also include determining whether a therapy will include an electrical block and/or electrical stimulation at or near the splanchnic nerve(s) (step 408). The determination may be made based on clinician input, patient input, and/or inputs received from one or more sensors 118. The decision to provide an electrical block versus an electrical stimulation may depend on which splanchnic nerve an electrode is implanted and based on current conditions around the patient. Current conditions may be determined based on sensor 118 inputs as well inputs received from the user interface 310.

The method 400 may also include determining whether the therapy will include an ablation of tissue of one or more splanchnic nerves (step 412). This particular step may be performed with assistance of clinician input. If the determination is made to ablate some or all of a particular splanchnic nerve, then a clinician may perform one or more surgical procedures using an ablation tool 204 to ablate some or all of one or more splanchnic nerves. As mentioned above, ablation may be performed by application of heat, RF energy, chemicals, or the like.

The method 400 continues by applying the therapy as determined in steps 408, 412 to the patient (step 416). Application of the electrical stimulation and/or blocking may continue after the surgical procedure in which ablation was performed. In some embodiments, the patient may have one or more electrodes implanted at or near a splanchnic nerve and electrical stimulation and/or blocking is provided to the patient as part of the therapy.

The method 400 may continue by optionally measuring subcutaneous nerve activity using one or more sensors (step 420). The sensors 118 used to collect sensor information and measure subcutaneous nerve activity may include implantable sensors, wearable sensors, environmental sensors, and the like. Subcutaneous nerve activity can also be measured by receiving patient and/or clinician inputs via the user interface 310. Measurements of the subcutaneous nerve activity may be processed by the feedback instructions 318 to determine if one or more adjustments need to be made to one or more parameters of the therapy (step 424). Examples of therapeutic parameters that may be adjusted in this step include, without limitation, signal frequency, peak current, duty cycle, application duration, etc. In addition to adjusting signal parameters, the adjustments made to the therapy may also include adjusting whether or not the patient is receiving a blocking signal and/or a stimulation signal. The adjustments may be made in real-time in response to sensor information just received or the adjustments may be made at spaced intervals. For example, the adjustments may be controlled by a clinician and may only be made at spaced intervals depending upon the clinician's availability and/or the clinicians judgment to make such adjustments, depending upon historical sensor information received from various sensor(s) 118.

The method 400 may continue with additional measurements of subcutaneous nerve activity (step 428). The sensor information measured in step 428 may be similar or identical to the sensor information measured in step 420. The ongoing measurement of sensor information to measure subcutaneous nerve activity may form a closed loop control system for modulating the patient's therapy over the course of time.

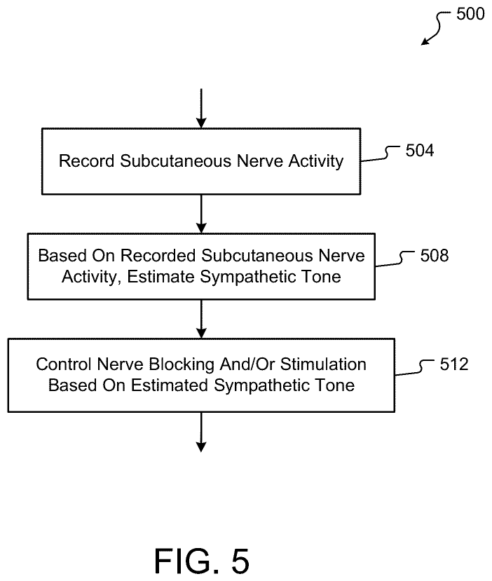

Referring now to FIG. 5, another method 500 will be described in accordance with at least some embodiments of the present disclosure. The steps of method 500 may be performed in the order depicted and described or may be performed in any other suitable order. It may also be possible to perform one or more steps of the method 500 simultaneously or concurrently without departing from the scope of the present disclosure. Moreover, steps of method 500 may be combined, interchanged, or supplemented with any other method steps depicted and described herein.

The method 500 begins by recording subcutaneous nerve activity (step 504). Subcutaneous nerve activity may be measured with one or multiple sensors 118. Examples of sensors 118 that may be used to measure subcutaneous nerve activity include, without limitation, wearable devices, implantable devices, user inputs to receive patient input regarding symptoms, user inputs to receive clinician input or feedback, location monitoring systems, tracking systems, combinations thereof, and the like.

Based on the measured subcutaneous nerve activity, the method 500 may continue by estimating a sympathetic tone of the patient (step 508). The sympathetic tone is a condition of a muscle when the tone is maintained predominantly by impulses from the sympathetic nervous system. The sympathetic tone may be elevated by catecholamine secretion, which can be driven by changes in patient motion, changes in physical activity, exposure to emotional stresses, etc. Sympathetic tone may also be estimated by measuring any number of indicators of subcutaneous nerve activity and is important in cardiac arrhythmogenesis. Electrical signals from the subcutaneous space of the thorax may come from multiple different sources, including low frequency motion artifacts, ECG generated by the heart, respiratory muscle activity and nerve activities. The nerve activities may include electrical activities from motor, sensory and autonomic nerves. High-pass filtering can eliminate the low frequency electrical activities including the ECG, leaving mostly the high frequency electrical activities (from sympathetic nerve fibers). Analyses of these recordings have shown that the electrical nerve signals in the subcutaneous space occurred simultaneously or nearly simultaneously with stellate ganglion (part of the sympathetic nervous system) nerve activity, leading to heart rate acceleration.

The method 500 then continues by controlling nerve blocking and/or nerve stimulation at the splanchnic nerve(s) based on the estimated sympathetic tone (step 512). In some embodiments, the estimates of sympathetic tone may be made using the feedback instructions 318 and the decisions on how to adjust nerve blocking and/or nerve stimulation may be made by the treatment instructions 316. It should be appreciated that step 512 may be performed as part of step 424 or may be performed to supplement step 424.

The present disclosure encompasses embodiments of the method 500 that comprise more or fewer steps than those described above, and/or one or more steps that are different than the steps described above.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A therapeutic system, comprising:
an implantable pulse generator configured to generate a current;
a plurality of electrodes configured to apply the current to one or more splanchnic nerves of a patient, wherein each of the plurality of electrodes comprises at least one of an anode or a cathode;
a processor; and
a memory storing data for processing by the processor, the data, when processed, causes the processor to:
receive sensor information describing subcutaneous nerve activity of the patient; and
adjust one or more parameters of the implantable pulse generator based on the subcutaneous nerve activity.

2. The system of claim 1, wherein the sensor information is received from at least one sensor provided on or implanted in the patient.

3. The system of claim 2, wherein the at least one sensor comprises at least one of a wearable sensor, an implantable sensor, and an environmental sensor, and wherein the sensor information is received via a wireless communication signal from the at least one sensor.

4. The system of claim 1, wherein the one or more parameters comprise at least one of frequency, peak current, duty cycle, and application duration.

5. The system of claim 1, wherein memory further stores data that, when processed by the processor, causes the processor to:
estimate a sympathetic tone based on the sensor information describing the subcutaneous nerve activity.

6. The system of claim 1, further comprising:
a user interface that allows at least one of the patient and a clinician to provide inputs to describe an aspect of the subcutaneous nerve activity.

7. The system of claim 1, wherein the one or more splanchnic nerves comprises at least one of a greater splanchnic nerve, a lesser splanchnic nerve, and a least splanchnic nerve.

8. The system of claim 1, wherein the one or more parameters of the implantable pulse generator are adjusted to increase a nerve blocking applied at the one or more splanchnic nerves.

9. The system of claim 1, wherein the one or more parameters of the implantable pulse generator are adjusted to decrease a nerve blocking applied at the one or more splanchnic nerves.

10. The system of claim 1, wherein the one or more parameters of the implantable pulse generator are adjusted to increase a nerve stimulation applied at the one or more splanchnic nerves.

11. The system of claim 1, wherein the one or more parameters of the implantable pulse generator are adjusted to decrease a nerve stimulation applied at the one or more splanchnic nerves.

12. The system of claim 1, wherein the sensor information is received from at least one of the plurality of electrodes.

13. A control subsystem for use in a therapeutic system, the control subsystem comprising:
a processor; and
a memory storing data for processing by the processor, the data, when processed, causes the processor to:
receive sensor information describing subcutaneous nerve activity of a patient; and
determine an electrical signal applied by an implantable pulse generator to a splanchnic nerve of the patient is to be adjusted based on the subcutaneous nerve activity; and
transmit a control signal to the implantable pulse generator thereby causing the implantable pulse generator to adjust the electrical signal applied to the splanchnic nerve.

14. The system of claim 13, wherein the electrical signal comprises at least one of a blocking signal that provides a nerve block and a stimulation signal that provides a nerve stimulation to the splanchnic nerve.

15. The system of claim 14, wherein the splanchnic nerve comprises at least one of a greater splanchnic nerve, a lesser splanchnic nerve, and a least splanchnic nerve.

16. The system of claim 13, wherein the sensor information is received from at least one sensor provided on or implanted in the patient.

17. The system of claim 13, wherein one or more parameters of the electrical signal are adjusted and wherein the one or more parameters include at least one of frequency, peak current, duty cycle, and application duration.

18. A method, comprising:
determining a first state of a therapy for a patient, wherein the first state of the therapy includes providing electrical signals to one or more splanchnic nerves of the patient;
receiving sensor information from one or more sensors;
based on the received sensor information, determining a subcutaneous nerve activity associated with the patient; and
based on the subcutaneous nerve activity, adjusting one or more parameters of the electrical signals to change the therapy from the first state to a second state.

19. The method of claim 18, wherein the first state of the therapy further comprises a unilateral ablation of at least a portion of the splanchnic nerve.

20. The method of claim 18, wherein the electrical signals are applied bilaterally to the one or more splanchnic nerves and wherein adjusting the one or more parameters comprises changing at least one of a frequency, peak current, duty cycle, and application duration for the electrical signals.
